(19) United States
(12) Patent Application Publication
(10) Pub. No.: US 2017/0136081 A1
(43) Pub. Date: May 18, 2017

(54) COMPOSITION AND METHOD FOR THE TREATMENT OF NEUROPATHIC PAIN

(71) Applicant: NEURAGEN CORP., Westbrook, ME (US)

(72) Inventor: Alexander McLellan, Ketch Harbour (CA)

(21) Appl. No.: 15/417,841

(22) Filed: Jan. 27, 2017

Related U.S. Application Data
(63) Continuation of application No. 13/576,922, filed on Aug. 2, 2012, now abandoned.
(60) Provisional application No. 61/300,677, filed on Feb. 2, 2010.

(51) Int. Cl. A61K 36/53; A61K 9/00

(52) U.S. Cl. CPC A61K 36/53; A61K 9/0053; A61K 9/0014; A61K 9/0043; A61K 2236/331

(57) ABSTRACT

The present invention provides a therapeutically effective composition and method of treatment of neuropathic pain utilizing extracts of Monarda, for example, Monarda fistulosa and related species, used alone or in a composition with a pharmaceutically acceptable carrier in a suitable dosage form.

### CROSS-REFERENCE TO RELATED APPLICATIONS

This is a continuation of U.S. patent application Ser. No. 13/576,922, filed Aug. 2, 2012, now pending, which is a 35 U.S.C. §371 National Stage application of International Application No. PCT/CA2011/050057, filed on Feb. 1, 2011, now expired, which claims priority to and the benefit under 35 U.S.C. §119(e) of U.S. Provisional Patent Application No. 61/300,677, filed Feb. 2, 2010, entitled “Composition and Method for the Treatment of Neuropathic Pain.” The disclosure of each of the prior applications is considered part of and is incorporated herein by reference in their entirety.

### FIELD OF THE INVENTION

The present invention pertains to the field of compositions comprising plant derived extracts and method of utilizing these extracts for the treatment of neuropathic pain. More particularly, the present invention pertains to compositions comprising extracts of Monarda.

### BACKGROUND

Neuropathic pain is pain caused by various types of nerve damage. Some examples of neuropathic pain conditions include, but are not limited to, diabetic peripheral neuropathy, herpes zoster, post herpetic neuralgia, trigeminal neuralgia, complex regional pain syndrome, reflex sympathetic dystrophy, migraine headache, phantom limb syndrome, neuropathic pain due to chronic disease (multiple sclerosis, HIV, etc), neuropathic pain due to trauma (causalgia), neuropathic pain due to impingement (i.e. sciatica, carpal tunnel, etc.), neuropathic pain due to drug exposure or toxic chemical exposure, neuropathic pain due to infection or post infection, neuropathic pain due to impaired organ function, neuropathic pain due to vascular disease, neuropathic pain due to metabolic disease, neuropathic pain due to cancer or cancer treatment, neuropathic pain due to autoimmune disease, neuropathic low back pain, neuropathic pain due to fibromylagia, and neuropathic pain with no known cause (idiopathic). In fact, neuropathic pain is most often diagnosed based on the symptoms, so that any pain is that is characterized by burning sensations and/or shooting pain and/or numbness and/or tingling and/or allodynia is typically considered neuropathic. Other characteristics of neuropathic pain include hyperpathia (greatly exaggerated pain sensation to stimuli), hyperesthesia (an increased sensitivity to normal stimulation), dysesthesia (unpleasant abnormal sensations as if damage is being done when this is not the case), and paresthesia (an abnormal sensation, such as “pins and needles”, whether spontaneous or evoked).

It is well known that nociceptive pain and neuropathic pain are caused by different mechanisms, and therefore respond to different treatment modalities. Nociceptive pain is mediated by receptors which are located in skin, bone, connective tissue, muscle and viscera. These receptors typically respond to noxious chemical, thermal and mechanical stimuli producing pain that is typically described as sharp, aching, throbbing, or gnawing. In contrast, neuropathic pain is produced by damage to, or pathological changes in, the peripheral or central nervous systems, typically producing pain that is described as “burning”, “electric”, “tingling”, and “shooting” in nature. Finally, nociceptive pain usually responds to opioids and non-steroidal anti-inflammatories (NSAIDS), whereas success treating neuropathic pain with these approaches has been limited. Conversely, agents employed to treat neuropathic pain, such as gabapentin, have little or no effect on nociceptive pain.

Current conventional pharmacologic strategies for treating neuropathic pain follow a number of different approaches as outlined below.

Antiarrhythmics: Certain antiarrhythmics have sodium-blocking activity. Low-dose IV lidocaine is sometimes used for temporary pain relief from peripheral nervous system injuries, including diabetic neuropathy and postherpetic neuralgia. However, IV lidocaine therapy requires constant monitoring of the patient's ECG and blood pressure to decrease the risk for seizures and arrhythmias.(Kalso, E Sodium Channel Blockers in Neuropathic Pain, Current Pharmaceutical Design, Volume 11, Number 23, September 2005, pp. 3005-3011(7))

Antidepressants: Both tricyclic antidepressants and serotonin reuptake inhibitors have been used to treat neuropathic pain. Numerous clinical trials demonstrate the safety and efficacy of TCAs when used to treat either diabetic neuropathy or postherpetic neuralgia, yet response rates have been low at approximately 33%. Amitriptyline was the first tricyclic used to treat neuropathy, and it is still widely prescribed. Amitriptyline has a high incidence of anticholinergic side effects, including delirium in elderly patients. TCAs also have proarrhythmic effects which limit their use in populations with abnormal EKG. Serotonin specific reuptake inhibitors (SSRIs) have demonstrated less consistent effects on neuropathic pain, relieving neuropathic pain in only one of seven patients. Serotonin noradrenaline reuptake inhibitors have fared slightly better with a response rate of one in every 4-5 neuropathic pain sufferers. (Sindrup, Soren H.; Otto, Marit; Finnerup, Nanna B. Jensen, Troels S. Antidepressants in the Treatment of Neuropathic Pain Basic & Clinical Pharmacology & Toxicology, Volume 96, Number 6, June 2005, pp. 399-409(11))

Anticonvulsants: Carbamazepine, phenytoin, gabapentin and lamotrigine have all been used to treat neuropathic pain. Inhibition of sodium channel blocking activity by agents such as carbamazepine, phenytoin, and lamotrigine is the proposed mechanism. Studies have shown the anticonvulsant gabapentin to be effective in painful diabetic neuropathy, mixed neuropathies, and postherpetic neuralgia. The most common adverse effects of anticonvulsants in general are sedation and cerebellar symptoms (nystagmus, tremor and incoordination). The most common side effects associated with gabapentin are asthenia, headache, dizziness and somnolence, and in some cases polyneuropathy. Lamotrigine was no better than placebo when used to treat neuropathic pain other than trigeminal neuralgia. (Jensen T S. Anticonvulsants in neuropathic pain: rationale and clinical evidence. Eur J Pain. 2002; 6 Suppl A:61-8)

NSAIDS: NSAIDS are not generally recommended first-line agents for treating neuropathic pain. Relief of neuropathic pain with nonsteroidal anti-inflammatory drugs (NSAIDs) is variable. (Kingery W S. A critical review of controlled clinical trials for peripheral neuropathic pain and complex regional pain syndromes. Pain 1997; 73:123-139)

Opioids: Treatment of neuropathic pain has with opioids has been controversial. Opioids were thought to be ineffective for treating neuropathic pain, but may be somewhat effective for patients who have failed other modalities. Short-term studies provide only equivocal evidence regarding the efficacy of opioids in reducing the intensity of neuropathic pain, while intermediate-term studies demonstrate significant efficacy of opioids over placebo. Reported adverse events of opioids are common and long-term efficacy, safety (including addiction potential), and effects on quality of life need to be further evaluated. Overall, neuropathic pain may be less responsive to opioids than other types of pain. (Elon Eisenberg, M D; Ewan D. McNicol, RPh; Daniel B. Carr, M D Efficacy and Safety of Opioid Agonists in the Treatment of Neuropathic Pain of Nonmalignant Origin JAMA. 2005; 293:3043-3052)

Other Agents:

Baclofen, which blocks both presynaptic and postsynaptic GABA B receptors, is used as a first-line agent to treat trigeminal neuralgia. The most common side effect is drowsiness, and there is concern about possible addictive effects. (Fromm G H. Baclofen as an adjuvant analgesic. J Pain Symptom Management 1994; 9(8):500-509)

Ketamine, an N-methyl-D-aspartic acid (NMDA) receptor antagonist, has garnered increased interest for treating neuropathic pain. Ketamine has been shown to relieve the symptoms of postherpetic neuralgia. However, ketamine causes sedation, slowed reaction times and hallucinations with long-term use. For this reason, it not currently recommended for use in chronic non-malignant pain. (Eide K, Stubhaug A, Oye I, Breivik H. Continuous subcutaneous administration of the N-methyl-D-aspartic acid (NMDA) receptor antagonist ketamine in the treatment of postherpetic neuralgia. Pain 1995; 61(2):221-8)

Dextromethorphan is also an NMDA antagonist. It has been used with some success to decrease pain in patients with diabetic neuropathy, but has not benefited those with postherpetic neuralgia, post stroke pain, or peripheral neuropathies other than diabetic. (Sindrup S H, Jensen T S. Efficacy of pharmacological treatments of neuropathic pain: an update and effect related to mechanism of drug action. Pain 1999; 83(3):389-400).

Topical Agents: Topical agents offer the advantage of local relief without systemic toxicity. Transdermal clonidine has been used with mixed results to treat diabetic neuropathy. Capsaicin cream, which contains an extract of chili peppers, is sometimes used to treat neuropathic pain. It may act on unmyelinated primary afferent nerves by depleting substance P. Depletion requires repeated and consistent use of capsaicin, and patient compliance can be an issue due to the common side effect of an intense burning sensation that decreases with consistent use. Overall, relief with capsaicin cream in clinical trials of neuropathic pain has been inconsistent.(4) Ketamine is a parenteral anesthetic agent that provides analgesic activity at sub-anesthetic doses. It is an N-methyl-D-aspartate (NMDA) receptor antagonist with opioid receptor activity. Controlled studies and case reports on transdermal ketamine demonstrate efficacy in neuropathic pain. (Kronenberg R H. Ketamine as an analgesic: parenteral, oral, rectal, subcutaneous, transdermal and intranasal administration. J Pain Palliat Care Pharmacother. 2002; 16(3):27-35)

U.S. Pat. No. 5,854,291 entitled “Pain Reliever and Method of Use” describes a composition containing capsaicin together with a second ingredient (such as a low concentration of plant extract from, for example, Monarda didyma) to treat pain, including neuropathic pain. The second ingredient is included in the pain reliever composition in order to reduce the irritating effects of capsaicin.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a composition and method for the treatment of neuropathic pain. The presently described and claimed method and composition of makes use of plant extracts from the genus Monarda for the treatment of neuropathic pain. Naturally occurring members of the genus can be utilized. Extracts from variants of Monarda, such as, but not limited to Monarda fistulosa L. var. menthaefolia can be used in the method and composition of the present invention.

In one preferred embodiment, a method for the treatment of neuropathic pain utilizing the naturally derived extracts of Monarda fistulosa (commonly known as bee balm, horsemint, and wild bergamot), is detailed. In another preferred embodiment, the extract of Monarda fistulosa is combined with other plant extracts and homeopathic ingredients to treat pain. In particular, the present invention provides a previously unavailable method for treating a range of neuropathies, through the administration to a mammal of plant extracts alone or in combination.

### BRIEF DESCRIPTION OF THE FIGURES

FIG. 1 is a graph illustrating the efficiency of application of the compound of the present invention to a 60 year old Caucasian male.

FIG. 2 is a graph illustrating the efficiency of application of the compound of the present invention to a 69 year old Caucasian female.

FIG. 3 is a graph illustrating the efficiency of application of the compound of the present invention to an 86 year old Caucasian female.

### DESCRIPTION OF THE INVENTION

It has now been found that Monarda extracts have valuable therapeutic properties. In particular, it has now been found that Monarda extracts, and compositions comprising a Monarda extract, have analgesic properties useful for treating neuropathic pain. Accordingly, the present application provides a method for treating the often unbearable and untreatable pain known as neuropathic pain, which is believed to be caused by aberrant nerve transmission due to damage to nerve tissue, by administering a plant extract from the genus Monarda, alone or in combination with other plant extracts or homeopathic ingredients.

Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

The term “plant material,” as used herein, refers to any part or parts of a specified plant taken either individually or in a group. Examples include, but are not limited to, leaves, needles, roots, bark, stems, buds, twigs, flowers, branches and the like.

The term “extract,” as used herein with reference to a specified plant, refers to a composition prepared by contacting plant material with a solvent following the procedures described herein. The extract can optionally be subjected to one or more separation and/or purification steps.

Monarda Extract

The Monarda extract used in the present method and composition can be prepared from any species or variant of a Monarda plant. The Monarda extract can be made from a single plant species, hybrid or variant or can be prepared using a combination of plant species, hybrids or variants. Furthermore, any plant material or combination of plant materials of the Monarda plant, for example, the Monarda leaves, flowers, buds, stems or trunk, or a mixture of those materials, are used in preparing the Monarda extract.

In accordance with one embodiment, the Monarda extract is prepared from any one of the following species, hybrids or variants, or any combination thereof:

Monarda fistulosa

Monarda Fistulosa L. var. menthaefolia

Monarda didyma

Hybrids of Monarda fistulosa and Monarda didyma

There are a variety of well known methods available to prepare extracts from raw plant materials, using processes such as distillation, solvent extraction, CO₂ extraction, cold pressing, expression, or enfleurage. The results of these extracts are typically known as “essential oils” or related mixtures such as volatile oils, ethereal oils, etc.

In a preferred embodiment, the Monarda extract is prepared using steam distillation.

Optionally, the Monarda extract used in the therapeutic method or use is a commercially available extract.

In order to confirm the therapeutic effect of the Monarda extracts, compositions comprising one or more Monarda extracts were administered to subjects having neuropathic pain. The subjects were then monitored for a reduction in neuropathic pain following administration as an indication of the therapeutic effect of the Monarda extract(s).

Therapeutic Applications

Based on the results of human clinical tests, Monarda plant extracts have now been found to have a beneficial effect on neuropathic pain conditions. Accordingly, the present application provides a therapeutic method and use of Monarda extract and a composition comprising a Monarda extract.

In one embodiment, the composition comprises an extract from Monarda fistulosa (for example, a steam distillate extract), or a blend containing this extract, alone or in combination with one or more a pharmaceutically acceptable diluents, carriers, excipients, or homeopathic ingredients. Thus, the present application provides a composition containing Monarda plant extracts that can be used together with one or more other active components in a range of relative amounts, or alone, and that is effective in the treatment of neuropathic pain.

Preferably, the composition of the present application comprises a Monarda extract in an amount of greater than about 0.5% by weight based on the total weight of the composition. Alternatively, the Monarda extract is present in an amount of greater than about 1.0% by weight, about 2.0% by weight or about 3.0% by weight, based on the total weight of the composition.

For administration to a mammal, the therapeutic composition can be formulated as a pharmaceutical formulation for topical, transdermal, oral, intranasal, rectal or parenteral administration or for administration by inhalation or spray. The composition can comprise the one or more Monarda extracts in dosage unit formulations containing one or more conventional non-toxic physiologically acceptable carriers, adjuvants and/or vehicles. The term parenteral as used herein includes subcutaneous injections, intravenous, intramuscular, intrathecal, intrasternal injections or infusion techniques.

In one embodiment, a therapeutically effective amount of the Monarda extract is administered orally to a subject (e.g., human).The pharmaceutical formulations can be in a form suitable for oral use, for example, as tablets, troches, lozenges, aqueous or oily suspensions, dispersible powders or granules, emulsion hard or soft capsules, or syrups or elixirs. Formulations intended for oral use can be prepared according to methods known in art for the manufacture of pharmaceutical compositions and can contain one or more agents selected from the group of sweetening agents, flavoring agents, coloring agents and preserving agents in order to provide palatable preparations.

Tablets contain the active ingredient in admixture with suitable non-toxic physiologically acceptable excipients including, for example, inert diluents, such as calcium carbonate, lactose, calcium phosphate or sodium phosphate; granulating and disintegrating agents, such as corn starch, or alginic acid, binding agents, such as starch, gelatine or acacia, and lubricating agents, such as magnesium stearate, stearic acid or talc. The tablets can be uncoated, or they can be coated by known techniques in order to delay disintegration and absorption in the gastrointestinal tract and thereby provide a sustained action over a longer period.

Pharmaceutical formulations for oral use can also be presented as hard gelatin capsules wherein the active ingredient is mixed with an inert solid diluent, for example, calcium carbonate, calcium phosphate or kaolin, or as soft gelatine capsules wherein the active ingredient is mixed with water or an oil based medium such as peanut oil, liquid paraffin or olive oil.

A syrup can be made by adding the active extract to a concentrated, aqueous solution of a sugar, for example sucrose, to which may also be added any necessary ingredients. Such accessory ingredient (s) can include flavorings, an agent to retard crystallisation of the sugar or an agent to increase the solubility of any other ingredients, such as polyhydric alcohol for example glycerol or sorbitol.

Oily suspensions may be formulated by suspending the plant extract(s) in a vegetable oil, for example, arachis oil, olive oil, sesame oil or coconut oil, or in a mineral oil such as liquid paraffin. The oily suspensions can contain a thickening agent, for example beeswax, hard paraffin or cetyl alcohol. Sweetening agents and/or flavoring agents may be added to provide palatable oral preparations. These formulations can be preserved by the addition of an anti-oxidant such as ascorbic acid.

Dispersible powders and granules suitable for preparation suitable for an aqueous suspension by the addition of water provide the active ingredient in admixture with dispersing or wetting agent, suspending agent and one or more preservatives. Suitable dispersing or wetting agents and suspending agents, sweetening, flavoring and coloring agents can also be present.

In another embodiment, a therapeutically effective amount of the Monarda extract is administered topically to the skin of a subject (e.g., human). The Monarda extract can be administered to the skin in the form of a cream, lotion, ointment, powder, spray, solution, gel, paste, serum, stick, foam, patch, face masks, etc. The MMP inhibitor may also be administered in a semi-solid dispersed system such as a nonionic, anionic, cationic, or gel network emulsion. Such an emulsion may be oil in water, water in oil, silicone in water, or water in silicone. The administration can be repeated in order to achieve the desired therapeutic effect.

In certain embodiments, the Monarda extract or composition thereof is administered at least once a day. The administration of the Monarda extract or compositions thereof can be continued for days, weeks, months, or indefinitely. The Monarda extract can be administered to an affected portion of the body, such as the hands or feet, or to the entire body.

To gain a better understanding of the invention described herein, the following examples are set forth. It should be understood that these examples are for illustrative purposes only. Therefore, they should not limit the scope of this invention in any way.

### EXAMPLES

### Example 1

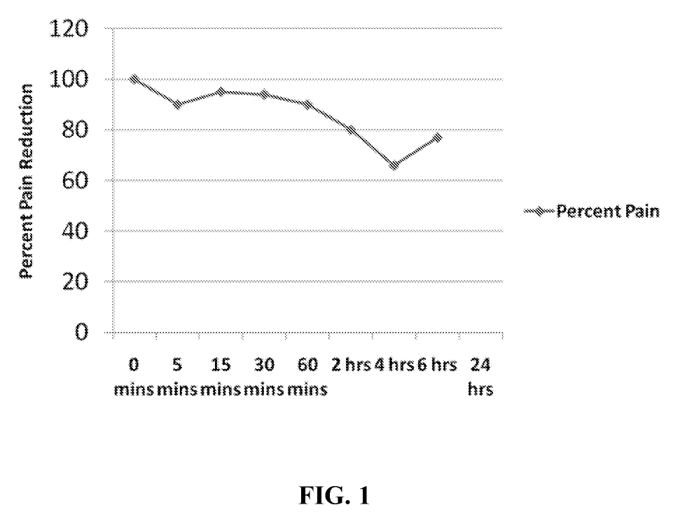

A 60 yr old Caucasian male with an approximately 2 yr history of Type II diabetes presented with chronic pain in both R&L hands and feet, burning, tingling and numbness in these areas. Current medications included Lyrica, glimepiride, vytorin, and exforge. Topical application of a thin film of extract of Monarda fistulosa (bee balm, wild bergamot, horsemint) resulted in pain reduction as shown in FIG. 1.

The Monarda fistulosa extract was produced using a steam distillation process known to those skilled in the art of essential oil production.

### Example 2

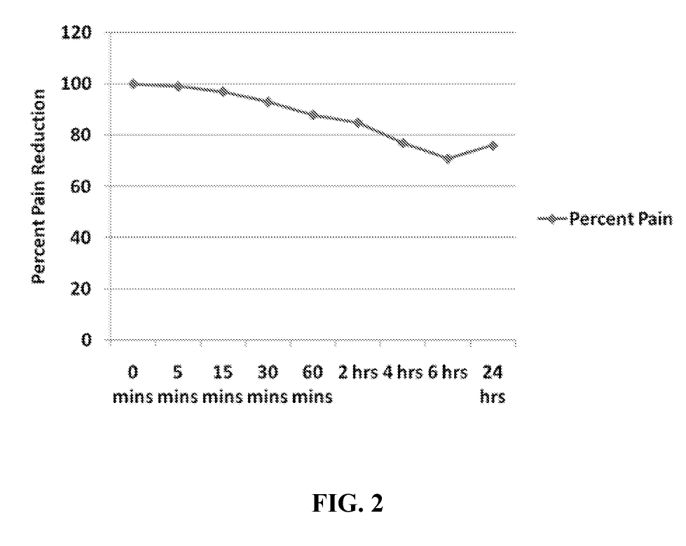

A 69 yr old Caucasian female with an approximately 1.5 yr history of chemotherapy induced neuropathy presented with chronic moderate to severe pain in both R&L hands and feet, burning, and numbness. Current medications included Arimidex. Topical application of a thin film of extract of Monarda fistulosa (the same extract as in Example 1) resulted in pain reduction as shown in FIG. 2.

### Example 3

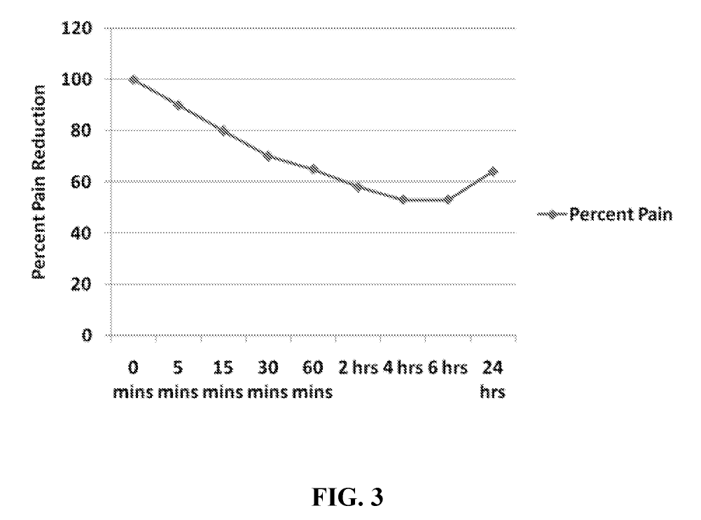

An 86 yr old Caucasian female with an approximately 0.5 yr history of idiopathic peripheral neuropathy presented with moderate pain in both R&L hands and legs (hips to feet) including burning, stinging, and numbness. Current medications included Lyrica, Synthyroid, and trimipramine. Topical application of a thin film of extract of Monarda fistulosa (the same extract as in Example 1) resulted in pain reduction as shown in FIG. 3.

### Example 4

A cream was produced containing 3.5% W/W Monarda fistulosa essential oil combined with 4% W/W essential oil of Lavender (Lavandula officinalis), 2.5% W/W essential oil of Bergamot (Citrus bergamia), 3% W/W essential oil of Eucalyptus (Eucalyptus globules), 2% W/W essential oil of Tea Tree (Melaleuca alternifolia), 5% W/W essential oil of Plai (Zingiber cassumunar Roxb.) as well as a blend of the homeopathic ingredients Hypericum Perforatum 12C, Aconitum Napellus 12C, Lycopodium Clavatum 12C, Phosphorous 12C, Rhus Toxicodendron 12C, Secale Cornutum 12C, Bryonia 3C, and Magnesium Phophate 3C at a total concentration of 1% W/W. Additional excipient ingredients included emulsifiers, thickening agents, surfactancts, preservatives and absorption enhancers were added as detailed in the table below:

| Ingredient | Amount (g) | % W/W | Function |
|---|---|---|---|
| Water | 80 | 40 | |
| Sodium Phosphate Dib [symbol] | 0.2 | 0.1 | emulsifier |
| Sodium Benzoate | 0.2 | 0.1 | preservative |
| Benzyl Alcohol | 2 | 1 | preservative |
| Polysorbate 80 | 4 | 2 | surfactant, |
| | | | emulsifier |
| Xanthan Gum | 0.4 | 0.2 | thickening |
| Lecithin | 2.77 | 1.38 | emulsifier |
| Isopropyl Myristate | 3.21 | 1.61 | absorption through |
| | | | skin |
| Sorbic Acid | 0.02 | 0.01 | preservative |
| Shea Butter | 25 | 12.5 | thickening |
| Beeswax | 17 | 8.5 | thickening |
| Sorbitan Monooleate | 4 | 2 | surfactant, emulsifier, |
| | | | dispersing |
| Propylene Glycol | 6 | 3 | solvent |
| Benzoic Acid | 0.2 | 0.1 | preservative |
| BHT | 0.2 | 0.1 | antioxidant |
| Cetyl Alcohol | 2.8 | 1.4 | thickening |

[symbol]

indicates data missing or illegible when filed

A 50 year old Caucasian male diagnosed with neuropathic low back pain, sciatica, and herniated disc at the L1 vertebra (based on MRI results) reported chronic pain of moderate intensity including burning pain, stabbing pain, painful cold sensations, numbness and tingling in the low back area. Within ten minutes of applying a thin film of the cream, he reported pain relief of 4/5 where 5 is complete relief and 0 is no pain relief. He preferred this method of pain relief to the oral pharmaceuticals he has been prescribed, which he cannot take during work hours due to the sedating effects.

### Example 5

A 53 year old Caucasian female diagnosed with neuropathic low back pain due to spinal stenosis reported chronic pain of moderate intensity (6-7/10) including burning pain, stabbing pain, numbness and tingling in the low back area. Within thirty n minutes of applying a thin film of the cream described in Example 4 to her low back area she reported pain relief of 5/5 where 5 is complete relief and 0 is no pain relief. She reported improved sleep when using the cream before bed, and improved ability to exercise when used during the day.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A method of treating neuropathic pain in a mammal by administering to the mammal a therapeutically effective amount of an extract from a plant of the genus Monarda or a variant thereof.

2. The method of claim 1, wherein the mammal is a human.

3. The method of claim 1, wherein the composition is a suspension, pill, gel, oil, cream, patch, spray or aerosol.

4. The method of claim 1, wherein the composition is administered orally.

5. The method of claim 1, wherein the composition is administered topically.

6. The method of claim 1, wherein the composition is administered intranasally.

7. The method of claim 1 wherein the composition is administered transdermally.

8. The method of any one of claims 1 to 7, wherein the Monarda extract is admixed with a pharmaceutically acceptable diluent, carrier, excipient, plant essential oil, or homeopathic ingredient.

9. The method of any one of claims 1 to 8, wherein the neuropathic pain is caused by or described as diabetic peripheral neuropathy, herpes zoster, post herpetic neuralgia, trigeminal neuralgia, complex regional pain syndrome, neuropathic low back, reflex sympathetic dystrophy, phantom limb syndrome, chronic disease (multiple sclerosis, HIV, etc), trauma (causalgia impingement (ie. sciatica, carpal tunnel, etc), drug exposure, toxic chemical exposure, a current infection, a past infection, impaired organ function, vascular disease, metabolic disease, cancer, cancer treatment, autoimmune disease or fibromylagia, or the neuropathic pain that has no known cause (idiopathic).

10. The method of any one of claims 1 to 8, wherein the neuropathic pain is characterized by burning sensations, shooting pain, numbness, tingling, allodynia, or any combination thereof.

11. The method of any one of claims 1 to 10, wherein the extract is prepared by a process that comprises steam distillation.

12. The method of any one of claims 1 to 11, wherein the extract is prepared from Monarda fistulosa.

13. The method of any one of claims 1 to 12, wherein the extract is administered in a composition comprising the extract in an amount of greater than 0.5% by weight.

14. The method of claim 13, wherein the extract is administered in a composition comprising the extract in an amount of greater than 1.0% by weight.

15. The method of claim 13, wherein the extract is administered in a composition comprising the extract in an amount of greater than 2.0% by weight.

16. The method of claim 13, wherein the extract is administered in a composition comprising the extract in an amount of greater than 3.0% by weight.

17. Use of an extract from a plant of the genus Monarda or a variant thereof for treating neuropathic pain in a mammal.

18. The use according to claim 17, wherein the mammal is a human.

19. The use according to claim 17, wherein the extract is formulated in a suspension, pill, gel, oil, cream, patch, spray or aerosol.

20. The use according to claim 17, wherein the extract is formulated for oral administration.

21. The use according to claim 17, wherein the extract is formulated for topical administration.

22. The use according to claim 17, wherein the extract is formulated for intranasal administration.

23. The use according to claim 17, wherein the extract is formulated for transdermal administration.

24. The use according to any one of claims 17 to 23, wherein the Monarda extract is admixed with a pharmaceutically acceptable diluent, carrier, excipient, plant essential oil, or homeopathic ingredient.

25. The use according to any one of claims 17 to 24, wherein the neuropathic pain is caused by or described as diabetic peripheral neuropathy, herpes zoster, post herpetic neuralgia, trigeminal neuralgia, complex regional pain syndrome, neuropathic low back, reflex sympathetic dystrophy, phantom limb syndrome, chronic disease (multiple sclerosis, HIV, etc), trauma (causalgia impingement (ie. sciatica, carpal tunnel, etc), drug exposure, toxic chemical exposure, a current infection, a past infection, impaired organ function, vascular disease, metabolic disease, cancer, cancer treatment, autoimmune disease or fibromylagia, or the neuropathic pain that has no known cause (idiopathic).

26. The use according to any one of claims 17 to 24, wherein the neuropathic pain is characterized by burning sensations, shooting pain, numbness, tingling, allodynia, or any combination thereof.

27. The use according to any one of claims 17 to 26, wherein the extract is prepared by a process that comprises steam distillation.

28. The use according to any one of claims 17 to 27, wherein the extract is prepared from Monarda fistulosa.

29. The use according to any one of claims 17 to 28, wherein the extract is formulated for administration in a composition comprising the extract in an amount of greater than 0.5% by weight.

30. The use according to claim 29, wherein the extract is formulated for administration in a composition comprising the extract in an amount of greater than 1.0% by weight.

31. The use according to claim 29, wherein the extract is formulated for administration in a composition comprising the extract in an amount of greater than 2.0% by weight.

32. The use according to claim 29, wherein the extract is formulated for administration in a composition comprising the extract in an amount of greater than 3.0% by weight.

33. An extract from a plant of the genus Monarda or a variant thereof for use in the treatment of neuropathic pain in a mammal.

34. The extract of claim 33, wherein the mammal is a human.

35. The extract of claim 33, wherein the extract is formulated in a suspension, pill, gel, oil, cream, patch, spray or aerosol.

36. The extract of claim 33, wherein the extract is formulated for oral administration.

37. The extract of claim 33, wherein the extract is formulated for topical administration.

38. The extract of claim 33, wherein the extract is formulated for intranasal administration.

39. The extract of claim 33, wherein the extract is formulated for transdermal administration.

40. The extract of any one of claims 33 to 39, wherein the Monarda extract is admixed with a pharmaceutically acceptable diluent, carrier, excipient, plant essential oil, or homeopathic ingredient.

41. The extract of any one of claims 33 to 40, wherein the neuropathic pain is caused by or described as diabetic peripheral neuropathy, herpes zoster, post herpetic neuralgia, trigeminal neuralgia, complex regional pain syndrome, neuropathic low back, reflex sympathetic dystrophy, phantom limb syndrome, chronic disease (multiple sclerosis, HIV, etc), trauma (causalgia impingement (ie. sciatica, carpal tunnel, etc), drug exposure, toxic chemical exposure, a current infection, a past infection, impaired organ function, vascular disease, metabolic disease, cancer, cancer treatment, autoimmune disease or fibromylagia, or the neuropathic pain that has no known cause (idiopathic).

42. The extract of any one of claims 33 to 40, wherein the neuropathic pain is characterized by burning sensations, shooting pain, numbness, tingling, allodynia, or any combination thereof.

43. The extract of any one of claims 33 to 42, wherein the extract is prepared by a process that comprises steam distillation.

44. The extract of any one of claims 33 to 43, wherein the extract is prepared from Monarda fistulosa.

45. The extract of any one of claims 33 to 44, wherein the extract is formulated for administration in a composition comprising the extract in an amount of greater than 0.5% by weight.

46. The extract of claim 45 formulated for administration in a composition comprising the extract in an amount of greater than 1.0% by weight.

47. The extract of claim 45 formulated for administration in a composition comprising the extract in an amount of greater than 2.0% by weight.

48. The extract of claim 45 formulated for administration in a composition comprising the extract in an amount of greater than 3.0% by weight.
